(19) United States
(12) Patent Application Publication
(10) Pub. No.: US 2024/0049950 A1
(43) Pub. Date: Feb. 15, 2024

(54) OPTICAL TRANSFORM CHARACTERISATION

(71) Applicant: Oxford University Innovation Limited, Oxford (Oxfordshire) (GB)

(72) Inventors: Martin J. BOOTH, Oxford (Oxfordshire) (GB); Raphael TURCOTTE, Oxford (Oxfordshire) (GB); Patrick S. SALTER, Oxford (Oxfordshire) (GB); Julian FELLS, Oxford (Oxfordshire) (GB)

(21) Appl. No.: 18/267,466

(22) PCT Filed: Dec. 17, 2021
(86) PCT No.: PCT/GB2021/053369

§ 371 Date: Jun. 15, 2023

(30) Foreign Application Priority Data
Dec. 18, 2020 (GB) 2020197.6

(51) Int. Cl. A61B 1/00; G01D 5/353; A61B 5/00

(52) U.S. Cl. CPC A61B 1/0017; G01D 5/35316; G01D 5/35367; A61B 5/0071

(57) ABSTRACT

A method of determining an optical transform imparted by a multimode optical fibre (300) is disclosed. The wherein the multimode fibre (300) comprises a proximal end, a distal end, and at least one modified region (260) between the proximal end and distal end. The modified region (260) is configured to transmit light toward the proximal end in response to light propagating through the multimode optical fibre (300) from the proximal end to the distal end. The method comprises coupling (101) forward propagating light into the proximal end of the multimode optical fibre; detecting (102), at the proximal end, backward propagating light transmitted from the at least one modified region in response to the forward propagating light; and determining (103) an optical transform from the detected backward propagating light.

### TECHNICAL FIELD

The present invention relates to the characterisation of an optical transform, and to devices that employ optical transforms. More particularly, the invention relates to optical transforms resulting from transmission through an optical fibre, and to devices configured to employ optical transforms that correct for transmission through an optical fibre.

### BACKGROUND

Multimode optical fibres (MMF) have a core diameter that is much larger that the wavelength of light that it the fibre is configured to carry, and can thereby support more than one mode of light propagation. MMF have a wide range of biomedical and industrial applications. Their small total diameter (typically 60-150 microns) provide access to hard-to-access locations and permits their insertion in biological tissue with limited disruption of physiological structures and functions. For example, minimally invasive micro-endoscopes based on MMF have been developed for in vivo brain imaging in the context of neuroscience studies. Such systems might be adapted for surgical guidance in other clinical applications (e.g. ear nose and throat applications).

A range of MMF applications, including the micro-endoscopes mentioned above, require spatially resolving optical signals for imaging or sensing. Contrast mechanisms for the optical signals in these applications include scattering and fluorescence, both in linear and nonlinear regimes, but could also be based on polarisation. Imaging with MMF can be implemented with widefield and point-scanning illumination and has been demonstrated with many modalities, for example: one-photon and multiphoton point-scanning fluorescence, confocal reflectance, coherent anti-Stokes Raman scattering, light-sheet, and SOFI (Super Resolution Optical Fluctuation Imaging. MMF are also used for microfabrication.

The location of the light source for illumination defines the proximal location and the position of the proximal end of the fibre. The proximal location refers to the space where the light source is located, and the proximal end comprises the MMF facet closest to the light source into which the illumination light is coupled. The distal location refers to the space at the other end of the fibre where the optical signal for imaging or sensing is generated, and the distal facet is the MMF facet at the distal end of the optical fibre that collects the optical signal. Between the proximal and distal end is the MMF, which serves as a waveguide to carry the illumination from the proximal end to the distal end and the optical signal from the distal end to the proximal end, which is optically coupled to a detector. The distal space (adjacent to the distal end) may be required to be free of optical elements (e.g. in a case where minimal invasiveness is required).

Optical effects occurring inside the MMF upon propagation cause the optical field to be transformed in an unpredictable, yet deterministic manner. This complex transformation T must be known for homomorphic mapping of information between the distal and proximal space with high spatial resolution as well as for spatially defined sensing in the distal space. Using a vectoral notation, T, known as the transmission matrix (TM), expresses the relationship between an input field at the proximal facet x and the resulting output field at the distal facet y′ and, reciprocally through time-reversal, an input field y at a distal facet and the resulting output field at the proximal facet x′:

y′=T_{λ}x (1)

y=T_{λ}^{T}x′ (2)

where the superscript T indicates the transpose operation and the wavelength dependence of T has been made explicit. A definition for T may be valid for a narrow spectral bandwidth. This reciprocity of the transmission matrix is illustrated schematically in FIG. 4.

The TM of an MMF is not only dependent on the illumination wavelength but also on the temperature and mechanical state of the fibre. This dependence on the fibre state S can be made explicit:

y′=T_{λ,S}x (3)

y=T_{λ,S}^{T}x′ (4)

In several dynamic systems, the MMF may be expected to be continually deformed, in ways that cannot necessarily be predicted. In such cases, the problem of determining the TM for each state is not tractable.

In most practical scenarios, a TM can be evaluated with the fibre in an initial state S₀ before its positioning (e.g. insertion in biological tissue). This approach can substantially simplify the evaluation of the TM upon continuous state changes (e.g. continuous deformation) using only proximal optical components, as the correction required to update the TM should also be continuous and small if evaluated in sufficiently close subsequent states. A correction matrix C_{λ,S}_{_{i-1→}}_{S}_{ᵢ} may be introduced to express the relationship between the TM of the current state Sᵢ to the previous state Sᵢ₋₁:

T_{λ,S}_{ᵢ}=C_{λ,S}_{_{i-1→}}_{S}_{ᵢ}T_{λ,S}_{ᵢ₋₁} (5)

where i∈N.

Existing Methods

Several methods have been proposed for evaluating the TM. Methods that require access to the distal end include:
i) Direct interferometeric evaluation¹
ii) Phase conjugation²
iii) Holographic optimisation³

Methods have also been proposed that do not require a camera or light source at the distal end of the MMF, including:
a) Plöschner et al⁴
b) Farahi et al⁵ ¹ C̆iz̆nàr, Tomàs̆, and Kishan Dholakia. “Shaping the light transmission through a multimode optical fibre: complex transformation analysis and applications in biophotonics.” Optics Express 19.20 (2011): 18871-18884.² Papadopoulos, Ioannis N., et al. “Focusing and scanning light through a multimode optical fiber using digital phase conjugation.” Optics express 20.10 (2012): 10583-10590.³ Di Leonardo, Roberto, and Silvio Bianchi. “Hologram transmission through multi-mode optical fibers.” Optics express 19.1 (2011): 247-254.⁴ Plöschner, M., Tyc, T. & C̆iz̆nàr, T. Seeing through chaos in multimode fibres. Nature Photonics 9, 529-535 (2015).
c) Gu et al⁶
d) Chen et al⁷
e) Gordon et al⁸
f) Li et al⁹

With the exception of Plöschner, all the other strategies require optical modulation beyond the distal facet. Plöschner requires that the TM, deformation and physical state of the MMF should be known, and allows a correction C_{λ,S}_{_{i-1→}}_{S}_{ᵢ} to be determined. In Plöschner, a theoretical approach is used to determine a TM for a bent fibre, based on prior knowledge of the shape of the bent fibre. Plöschner therefore is of limited practicality in contexts where the deformation of the fibre is not known or predictable. Furthermore, while this approach works well for a short fibre (<10 cm) (which is fine for rodent applications) it is limited in how well it can deal with longer fibres with more complex conformation (including compound curvature). A 10 cm fibre length is not sufficient for many clinical and industrial applications. The calculations are also complex.

In Farahi a reference light is delivered to the distal end of the MMF using a single mode fibre that runs in parallel with the multimode fibre (either concentrically, in a double clad fibre, or via a coupling prism in the case of a laterally offset single mode fibre). The reference light is diffracted by a holographic pattern recorded in a polymer layer at the distal end of the multimode fibre and reflected back into the multimode fibre. The diffracted reference light reflected back into the multimode fibre approximates an object point source, or virtual beacon. The proximal intensity distribution of this virtual beacon (after transmission through the multimode fibre) is measured, and correlated to a data bank of patterns corresponding to different fibre ⁵ Farahi, S., Ziegler, D., Papadopoulos, I. N., Psaltis, D. & Moser, C. Dynamic bending compensation while focusing through a multimode fiber. Optics Express 21, 22504-22514 (2013).⁶ Gu, R. Y., Mahalati, R. N. & Kahn, J. M. Design of flexible multi-mode fiber endoscope. Optics Express 23, 26905-26918 (2015).⁷ Chen, H., Fontaine, N. K., Ryf, R., Neilson, D. T. & Winzer, P. Remote Spatio-Temporal Focusing over Multimode Fiber Enabled by Single-Ended Channel Estimation. Ieee Journal of Selected Topics in Quantum Electronics 1-1 (2020) doi:10.1109/jstqe.2020.2979241.⁸ Gordon, G. S. D. et al. Characterizing Optical Fiber Transmission Matrices Using Metasurface Reflector Stacks for Lensless Imaging without Distal Access. Phys Rev X 9, 041050 (2019).⁹ Li, S., Horsley, S. A. R., Tyc, T., Cizmar, T. & Phillips, D. B. Guide-star assisted imaging through multimode optical fibres. Arxiv (2020). conformations. The data bank also includes the TM associated with each fibre conformation. This enables the TM for the fibre conformation to be determined from the detected virtual beacon at the proximal end of the fibre.

In Gu a thin reflective surface with a checkboard pattern is placed at the distal end of the MMF. Each square of the checkerboard pattern has a different reflection coefficient, with the result that spatial modulation of the amplitude of the reflectance signal is achieved. Light reflected from the checkerboard pattern is imaged at the proximal end of the fibre by a camera, and the resulting pattern used to determine an updated TM from an initial TM (that may be predetermined with access to both ends of the fibre, using and if the methods i) ii) or iii) mentioned above).

In Chen many single mode fibres are placed at the distal end, and the approach is somewhat equivalent to Gu. This is not practical in biomedical applications and other industrial applications, where it is not possible to place a lot of optical equipment at the distal end of the MMF.

In Gordon an extension of the method proposed by Gu is described, in which thin reflectors are stacked on the distal end of the MMF, and further encoding is done in each layer based on polarisation and wavelength. This is a more general approach, as it extends to non-unitary MMF. The approach is essentially the same as Gu, but instead of requiring N references, corresponding to the number of elements of the TM diagonal, N×N references are needed, which enables determination of the full TM.

Farahi, Gu, and Gordon all require assembly of micro-optics—which involves multiple complex components at the distal end (e.g. coupled to the distal facet of the MMF). This is complex and not-straightforward.

Li discloses the use of a guide star, which could be a fluorescent bead attached to the tip of the fibre. The TM may be found by an optimisation algorithm that determines an approximate transmission matrix by optimising constructive interference onto the guide star. The TM determined using this method is valid over an isoplanatic patch, the size of which is determined by the number of propagation invariant modes that have power at the position of the guide star. In practice, a guide star at the interface between the core and cladding was found to be optimal.

Each of the existing methods for compensating for the transfer function of a MMF is complex in practical implementation. Methods and devices that make it easier to determine a MMF TM are desirable, particularly if they facilitate determining corrections for movement/deformation of the MMF.

### SUMMARY

According to a first aspect, there is provided a method of determining an optical transform imparted by a multimode optical fibre, wherein the multimode fibre comprises a proximal end, a distal end, and at least one modified region between the proximal end and distal end, the modified region configured to transmit light toward the proximal end in response to light propagating through the multimode optical fibre from the proximal end to the distal end, the method comprising:
coupling forward propagating light into the proximal end of the multimode optical fibre;
detecting, at the proximal end, backward propagating light transmitted from the at least one modified region in response to the forward propagating light;
determining an optical transform from the detected backward propagating light.

Determining the optical transform may comprise:
i) determining a transmission matrix of the multimode optical fibre, defining a relationship between an input field at a proximal facet and the resulting output field at a distal facet; and/or
ii) determining a correction matrix for correcting a predetermined transmission matrix (for example to correct changes in conformation of the optical fibre).

The transmission matrix comprises a matrix of complex coefficients relating the input field amplitude and phase of each of a plurality of input modes to the output field amplitude and phase of each of a plurality of output modes. The correction matrix may comprise a set of corrections for correcting a transmission matrix (e.g. as defined in equation (5))

Providing a modified region in the MMF is a more elegant solution that prior art methods that involve optics appended to the distal facet of the MMF, or that rely on optical components arranged about the distal end of the MMF.

The at least one modified region may comprise at least one fibre Bragg grating. A fibre Bragg grating can be written in a MMF, for example using laser micromachining.

The at least one modified region may comprise at least one fluorescent colour centre. A fluorescent colour centre will emit light at a different wavelength to the illuminating light, which may make it possible to perform sensing using the MMF at the same time as determining the optical transform of the MMF (and using the optical transform to optimise/correct the sensing).

The at least one modified region may comprise one or more modified regions disposed in the fibre core and/or fibre cladding.

There may be a plurality of modified regions, with at least some of the modified regions at different lateral positions and/or at different longitudinal positions between the proximal and distal end.

The at least one modified region may comprise a plurality of fibre Bragg gratings, and at least some of the fibre Bragg gratings may have different: period, reflectivity and/or orientation/polarisation

The at least one modified region may comprise a one or more chirped fibre Bragg gratings.

The forward propagating light may be first forward propagating light field and the backward propagating light may be a first backward propagating light field, and the method may further comprise:
transmitting a second forward propagating light field for sensing/imaging a scene adjacent to the distal end;
detecting a second backward propagating light field transmitted through the fibre resulting from the second forward propagating light field, the method further comprising using the optical transform determined from the first backward propagating light field to correct/optimise an image formed using the second backward propagating light field.

Correcting/optimising the image may comprise controlling an active optical element to modify the second forward propagating light field.

The active optical element may comprise a spatial light modulator configured to modify the spatial distribution of phase of the second forward propagating light field

The method may comprise performing point scanning microscopy using a plurality of second forward propagating light fields.

Correcting/optimising the image may comprise computationally reconstructing an image from the results of detecting the second backward propagating light field.

The first forward propagating light field and the second forward propagating light field and/or the first backward propagating and the second backward propagating light field are multiplexed, so that the optical transform can be updated without interrupting imaging of the scene.

The multiplexing may comprise wavelength multiplexing and/or temporal multiplexing.

The modified region may be is formed by laser machining

The laser micromachining may be is performed using adaptive optics, which modify wavefront properties of a laser system to counteract the effects of aberration on laser focus

Determining on optical transform may comprise determining a correction matrix to take account of fibre deformation/conformation, wherein determining the optical transform comprises multiplying the correction matrix with an uncorrected transmission matrix.

The uncorrected transmission matrix may be determined by detecting, at the distal end of the fibre, forward propagating light coupled into the optical fibre at the proximal end of the fibre.

The method may further comprise correcting the transmission matrix for temperature determined from a modified region that comprises a fibre Bragg grating.

According to a second aspect, there is provided apparatus for obtaining information for correcting an optical transform imparted by a multimode optical fibre, comprising:
a multimode fibre comprising
   a proximal end,
   a distal end, and
   at least one modified region between the proximal end and distal end, the modified region configured to transmit light toward the proximal end in response to light propagating through the multimode optical fibre from the proximal end to the distal end,
a light source coupled to the proximal end and configured to transmit forward propagating light into the optical fibre;
a detector coupled to the proximal end and configured to detect backward propagating light transmitted from the at least one modified region in response to the forward propagating light.

The apparatus may comprise a processor configured to determine the optical transform from the detected backward propagating light.

The apparatus according to the first aspect may be configured to perform the method according to the first aspect, including any of the optional features thereof.

The forward propagating light may be a first forward propagating light field and the backward propagating light is a first backward propagating light field; and
the light source is configured to transmit a second forward propagating light field for imaging a scene adjacent to the distal end;
the detector is configured to detect a second backward propagating light field transmitted through the fibre resulting from the second forward propagating light field; and
the apparatus is configured to use the detected backward propagating light field to correct an image formed using the second backward propagating light field.

The apparatus may be configured to use an optical transform determined from the detected first backward propagating light field to correct the image formed using the second backward propagating light field

According to a third aspect, there is provided an endoscope comprising the apparatus of the second aspect, including any optional features thereof.

The features (including optional features) of any aspect may be combined with those of any other aspect, as appropriate.

### BRIEF DESCRIPTION OF THE DRAWINGS

Example embodiments will be described, by way of example only, with reference to the drawings, in which:

FIG. 1 shows a method according to an embodiment;

FIG. 2 shows a schematic of an apparatus according to an embodiment;

FIG. 3 shows a schematic of an apparatus according to an embodiment, employing wavefront control and a multimode fibre for imaging or sensing;

FIG. 4 illustrates the reciprocity of the transmission matrix;

FIGS. 5 and 6 show a modified region comprising a Bragg grating inscribed in a fibre;

FIGS. 7 and 8 show a modified region comprising a point structure inscribed in a fibre;

FIGS. 9 and 10 show Bragg gratings, or other structures, inscribed at different radial and azimuthal locations in a fibre.

FIG. 11 shows Bragg gratings, or other structures, inscribed at different longitudinal locations in a fibre;

FIG. 12 shows Bragg gratings, or other structures, inscribed at different locations along a Fibre for characterising conformational changes;

FIG. 13 shows how multiple structures can be inscribed and stacked within a segment of the fibre that may be kept rigid (e.g. by addition of external tubing or by the specimen) at the distal end of a fibre;

FIG. 14 shows amplitude multiplexing of the backward propagating light from the modified regions by using Bragg gratings of different length (reflectivity);

FIG. 15 shows spectral multiplexing of the backward propagating light from the modified regions by using Bragg gratings of different periodicity;

FIG. 16 shows polarisation multiplexing of the backward propagating light from the modified regions by using Bragg gratings of different shapes; and

FIG. 17 shows a two-dimensional array of Bragg gratings of varying lengths at different locations for spatially modulating the amplitude of the reflectance signal;

FIG. 18 illustrates that two-dimensional arrays of Bragg gratings operating at different wavelengths can be stacked to further multiplex the reflected signal; and

FIG. 19 illustrates an embodiment in which the modified region comprises a Bragg near the edge of the core, which may be suitable for serving as a guide star from which the total reflected intensity can be maximised.

### DETAILED DESCRIPTION

Referring to FIG. 1, a method of determining an optical transform imparted by a multimode optical fibre is shown. The multimode fibre comprises a proximal end, a distal end and at least one modified region between the proximal end and distal end. The modified region is configured to transmit backward propagating light toward the proximal end of the fibre in response to forward propagating light transmitted through the optical fibre from the proximal end towards the distal end. The modified region may, for example, comprise a reflective element (such as a fibre Bragg grating) or a fluorescent element. The modified region may be formed by laser machining of the optical fibre, and may be disposed in the fibre core or cladding.

At step 101, forward propagating light is coupled into the proximal end of the MMF. The forward propagating light will reach the modified region in the MMF, and the modified region will cause a backward propagating light signal in response to the forward propagating light (e.g. by fluorescing in response to the forward propagating light, or by reflecting a portion of the forward propagating light).

At step 102, the backward propagating light arising from the interaction of the forward propagating light with the modified region is detected, after it exists from the proximal end of the fibre. A beam splitter may be used to both transmit the forward propagating light into the fibre and to detect the backward propagating light from the modified region. The backward propagating light from the modified region may be imaged, to produce an intensity distribution of the backward propagating light at the proximal end of the fibre.

At step 103, an optical transform TM is determined from the results of detecting the backward propagating light exiting the optical fibre at the proximal end. For example, the intensity distribution of the backward propagating light from the modified region may be used to determine a TM that includes conformation effects, or to determine a correction matrix for correcting a predetermined TM for changes in conformation of the optical fibre.

The detected backward propagating light from the modified region of the fibre can be used to characterise the optical transform imparted by the fibre without access to the distal end. The backward propagating light originates from one or more known positions in the optical fibre (corresponding with one or more modified regions disposed in the optical fibre). The techniques described in the background section can readily be used to characterise the TM in a way that takes into account conformation changes in the fibre to enable imaging using the MMF.

The TM can be used in several ways, which can be grouped into two categories.

The first category encompasses methods in which a distal plane is illuminated with a sequence of non-uniform wide-field illuminations. A distal image can be reconstructed computationally for each image recorded with a proximal detector using the TM. In such an approach, the detector may be used to both determine the TM from the backward propagating light from the modified region, and to obtain the image data from which the distal image is computationally determined using the TM. In some embodiments the determination of the TM may be temporally multiplexed with the acquisition of imaging data (from which the image is determined), for example between each frame, or periodically, depending on how much movement of the MMF is anticipated during imaging (e.g. every 5 imaging frames, or every 10 imaging frames etc). The acquisition of multiple images enables averaging out the non-uniformities in the illuminations due to MMF optical effects. In some embodiments, different light sources and/or different detectors may be used to determine the TM and perform imaging at the same time, using wavelength multiplexing of: the forward propagating light used to interrogate the at least one modified region and the forward propagating light used to image a scene at the distal plane; and/or of the backward propagating light from the one or more modified region and the backward propagating light from the distal plane.

The second category uses the TM, not after data acquisition, but instead to modify the illumination. A wavefront shaping device (such as a spatial light modulator) may be used to modulate the input light field that is used for imaging, so as to shape the light at the distal plane. One implementation of this is to generate a diffraction-limited focal point in the distal plane. This point enables sensing or optical manipulation (e.g. nonlinear photo-polymerisation) in a spatially defined and limited location. The point can also be translated by updating the input field modulation in a fashion equivalent to raster-scanning for realising point-scanning microscopy. Suitable wavefront shaping devices include liquid crystal spatial light modulators and digital micromirror devices.

The methods of characterising the TM described herein are applicable to both categories of using the TM.

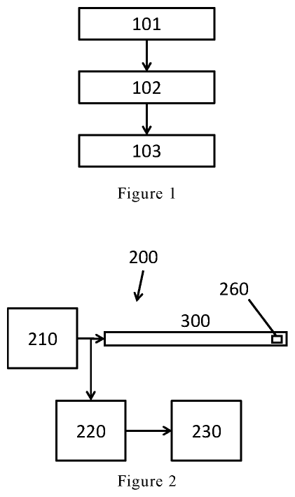

FIG. 2 illustrates, in simplified form, an apparatus 200 according to an embodiment, comprising a multimode optical fibre 300, light source 210, detector 220 and processor 230.

The multimode optical fibre 300 comprises a modified region 260 near to the distal end thereof (e.g. within 1 cm, or 5 mm or 2 mm or closer), which may comprise a fibre Bragg grating. In some embodiments the modified region may be closer than 1 mm from the distal end (e.g. within 0.5 mm or 0.2 mm). The modified region 260 may be written using laser machining, which may employ active optics to modify the wavefront properties of the laser to correct for aberration of the laser focus (for example, as described in US2020/166698). In some embodiments there may be a plurality of modified regions, examples of which will be described in more detail below.

The light source 210 is optically coupled to the proximal end of the fibre 300, and transmits forward propagating light into the optical fibre 300. The forward propagating light interacts with the modified region 260, which results in backward propagating light.

The detector 220 is configured to detect the backward propagating light from the interaction of the forward propagating light with the modified region 260, for example by imaging the resulting pattern of light at the proximal end of the fibre 300.

The results from the detector 220 are provided to processor 230 (e.g. in the form of an image of the spatial distribution of light intensity), which determines the TM of the MMF 300 therefrom. Depending on the specific arrangement and nature of the modified region (or regions), any of techniques a) to f) described in the background section may be employed.

In embodiments employing the technique of Plôschner there may be one or many Bragg gratings 260, which may be used to characterise conformal changes in the MMF 300. One way to do this is to use the Bragg gratings as strain gauges. For example a distribution of Bragg gratings around the perimeter of the fibre can be used to detect bending of the fibre in different directions. As the Bragg grating is strained, the Bragg wavelength defined by the grating spacing changes, which can be detected. Gratings with different Bragg wavelengths can be used, enabling wavelength multiplexed interrogating of the strain gauges. The conformal changes may be used to determine a correction matrix C_{λ,S}_{_{i-1→}}_{S}_{ᵢ} for updating a predetermined TM (as described in Plöschner).

In some embodiments, the modified region may comprise a single Bragg grating, and the reflected pattern imaged proximally. The intensity distribution may be correlated with a plurality of predetermined intensity distributions in a databank to determine a corresponding TM, following the technique described in Farahi (with the Bragg grating serving as a virtual beacon). A single grating may be sufficient to characterise a correction matrix (e.g. for relatively small changes in conformation of the fibre). Multiple gratings may be used to characterise a correction matrix for more significant conformation.

In some embodiments, multiple Bragg gratings can be written at different locations in the fibre. A checkboard reflector following the approach of Gu can be generated by employing a similar spatial distribution of Bragg gratings near to the distal tip of the MMF 300, with spatial modulation of the reflectance signal achieved by using Bragg gratings with different lengths (i.e. a different number of periodic variations in refractive index) with longer Bragg gratings providing higher reflectance at the Bragg wavelength.

The multiple Bragg gratings can be probed (by the forward propagating light from the light source 210) at different times, or the light may be wavelength multiplexed. The light incident on the Bragg gratings may be shaped by using wavefront control at the light source 210.

The approach set out in Gordon can be followed by employing Bragg gratings with different grating periods, and hence having different Bragg wavelengths. In some embodiments, two-dimensional arrays of Bragg gratings may be stacked (longitudinally near the distal end of the fibre), with each layer in the stack having a different Bragg wavelength. Different layers gratings may be configured with different diattenuation by changing the orientation of the gratings used to define them.

The approach set out in Li can be followed by disposing a modified region (Bragg grating or fluorescent centre) to serve as s guide star, from which the TM can be found that optimises constructive interference onto the guide star. The modified region may be disposed at the interface between the core and the cladding, to maximise the size of the isoplanatic patch. Multiple modified regions may be used to tile multiple isoplanatic patches into a single image. Each guide star may partial information of the transmission matrix. The transmission matrix basis can be expressed to make the information obtained from each guide star independent. This is also a good strategy for minimising the number of required guidestars for determining the transmission matrix

Machine learning approaches can also be used. In one example, an image of the back-propagating light from the one or more modified regions is provided to a convolutional neural network (CNN). The CNN may be trained using controlled fibre deformations and corresponding TMs that have been determined with access to both ends of the fibre (for example using methods i) to iii) disclosed in the background section). The CNN may consequently be able to determine a TM directly from a detected image (spatial distribution of intensity) of the back propagating light at the proximal end of the fibre. In some embodiments, the CNN may be provided with image data corresponding with different wavelengths (e.g. reflected from gratings with different period) and with different polarisation states (e.g. reflected from gratings with differently oriented periodic variations in refractive index). The CNN may be configured to determine a correction matrix for correcting a predetermined TM corresponding with a specific conformation of the MMF (e.g. a straight fibre, or a fibre in a neutral starting position of a particular instrument).

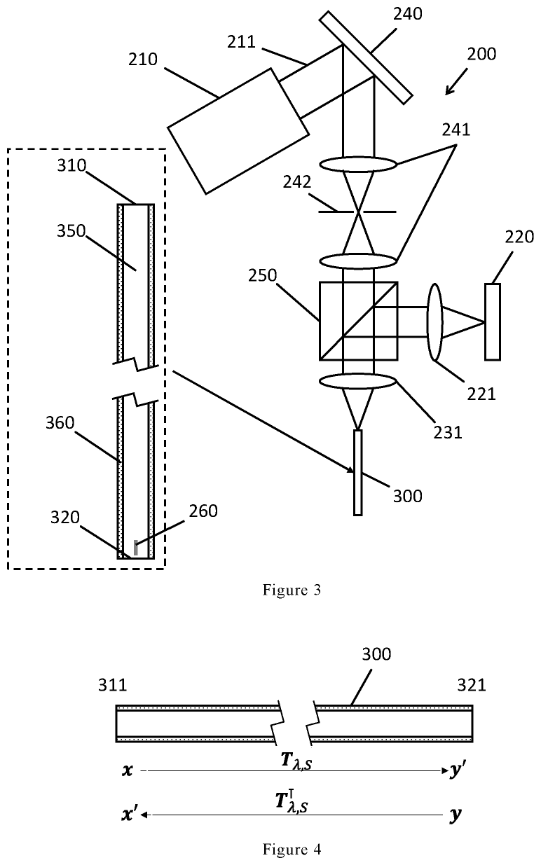

FIG. 3 shows a further example embodiment of an apparatus 200 according to an embodiment, comprising: light source 210, wavefront shaping device 240, relay lenses 241, aperture 242, beam splitter 250, imaging lens 221, detector 220, coupling lens 231 and multimode optical fibre (MMF) 300.

The MMF 300 comprises a proximal end 310, distal end 320, core, 350, cladding 360 and modified region 260 near to the distal end 320.

The light source 210 is optically coupled to the proximal end 310 of the MMF 300 via the wavefront shaping device 240, relay lenses 241, aperture 242, beam splitter 250 and coupling lens 231. A light beam from the light source 210 is modified by the wavefront shaping device 240, and then coupled into the proximal facet (at the proximal end) of the MMF 300 via the relay lenses 241, aperture 242 and coupling lens 231. The wavefront shaping device 240 may comprise a spatial light modulator, for example employing liquid crystal modulators that are configured to adjust the spatial distribution of phase of the light. The wavefront shaping device 240 may be configured to shape light incident on the modified region, or to perform imaging by shaping light incident on a distal plane (as described above).

The beam splitter 250 is configured to couple forward propagating light from the light sources 210 into the proximal end 310 of the MMF 300, and to coupled backward propagating light emerging from the proximal end 310 of the MMF to the sensor 220, via the imaging lens 221. The detector 220 may comprise a focal plane array, at which an image of the backward propagating light is formed. The detector 220 may be used to detect the backward propagating light from the modified region 260 for characterisation of the TF of the MMF 300, and may also be used to form an image of a scene adjacent to the distal end 320 of the MMF 300 (as described above) using the TF to correct the image. Using the TF to correct/optimise the image may comprise generating a point scanning illuminating light field at the distal scene using the wavefront shaping device 240, and/or may comprise computationally reconstructing an image of the distal scene employing the TF (from one or more images taken from the detector 220).

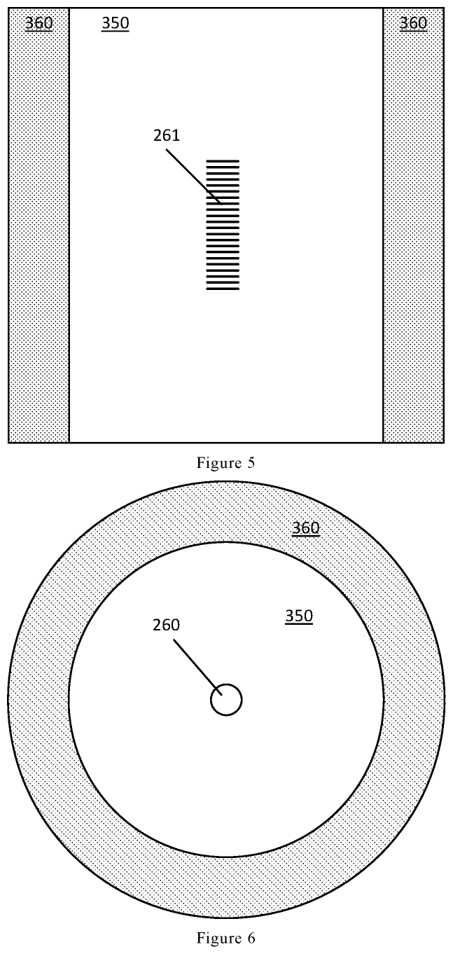

FIGS. 5 and 6 show an end view and side view respectively of an example of a modified region according to an embodiment comprising a Bragg grating 261 written near the distal end of a MMF. In this example embodiment, the Bragg grating 261 is centrally located, on the axis of the optical fibre core 350.

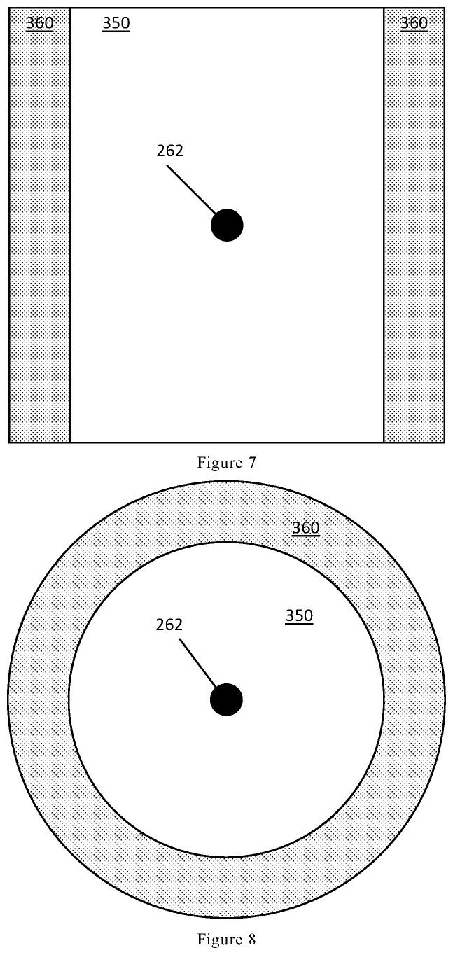

FIGS. 7 and 8 show an end view and side view respectively of an example of a modified region according to an embodiment comprising a point structure 262, such as a fluorescent centre written near the distal end of a MMF. In this example embodiment, the point structure 262 is centrally located, on the axis of the optical fibre core 350.

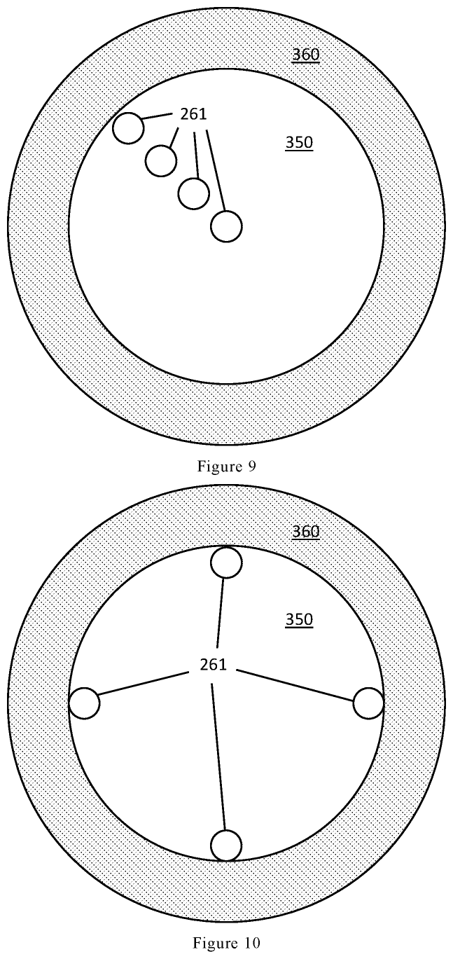

FIG. 9 shows an example embodiment in which a plurality of modified regions in the form of Bragg gratings 261 are disposed in the core 350 near the distal end of the MMF. The Bragg gratings 261 are arranged in a one dimensional array along a radius of the fibre core, with different gratings 261 arranged at different radial positions.

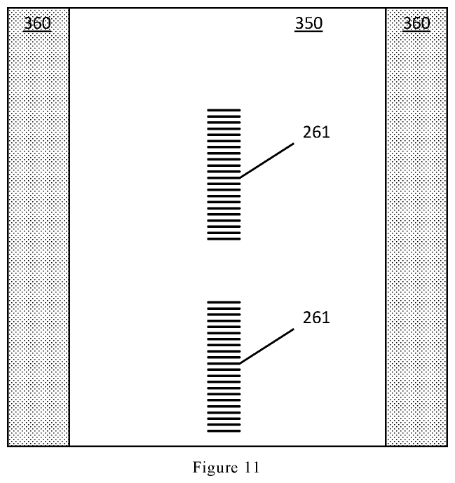

FIG. 10 shows an example embodiment in which a plurality of modified regions in the form of Bragg gratings 261 are disposed at different circumferential locations at the perimeter of the fibre core 350, near a distal end of the MMF. In the example the gratings 261 are equally spaced around the circumference of the fibre core 350, but this is not essential.

FIG. 11 shows a side view of multiple modified regions comprising Bragg gratings 261, disposed on the longitudinal axis of the MMF at different longitudinal positions near to the distal end of the MMF. Although a single Bragg grating at each longitudinal position is shown, there may be more than one Bragg grating at each longitudinal position, and the modified regions may be dispersed both laterally (e.g. in different radial and circumferential positions at a particular longitudinal direction) and longitudinally.

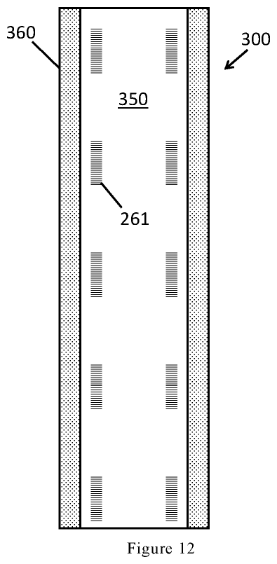

FIG. 12 illustrates a side view of multiple modified regions comprising Bragg gratings 261 written near to the edge of the core 350. The Bragg gratings 261 may be evenly distributed along the length of the MMF, with each longitudinal location having a evenly spaced circumferential array of Bragg gratings 261 disposed near to the edge of the core 350 (or in the cladding 360). In such an arrangement each grating is sensitive to bending (with bending at each longitudinal location imparting different strain at different circumferential locations, depending on the direction of bending, which can be detected by a changed in the Bragg wavelength of each grating. Such an arrangement allows conformal changes in the MMF to be sensed. The conformal changes can be used in a calculation as disclosed in Plöschner to determine a correction matrix for updating a TM (which may have been predetermined by techniques i) to iii), for example with access to both proximal and distal ends of the MMF.

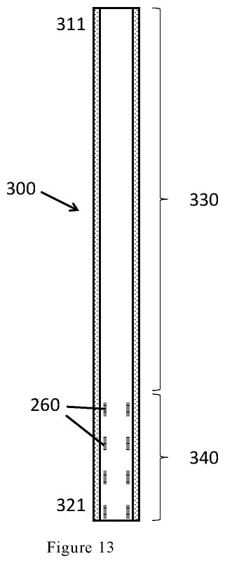

FIG. 13 shows an MMF 300 according to an embodiment in which multiple modified regions 260 can be provided in a distal end region 340 of the MMF 300. The multiple modified regions may comprise Bragg gratings, and may be provided at a series of longitudinal positions, with each longitudinal position comprising a plurality of gratings (e.g. as shown in any of FIGS. 5, 6, 9 and 10). The distal end region 340 of the MMF may be kept substantially free from conformation changes, either by a support structure (such as a rigid support tube) or by the specimen being imaged (e.g. by tissue surrounding the MMF, in the case of invasive endoscopy). The modified regions 260 in the distal end region 340 of the MMF may be used to determine a TM (or a correction matrix) that takes account of conformation changes in the rest of the MMF 330.

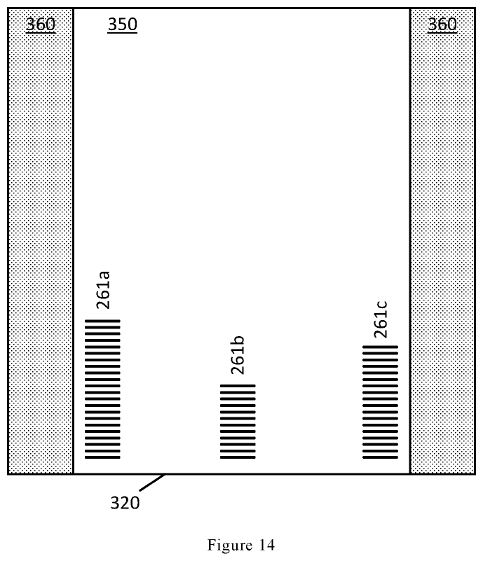

FIG. 14 shows a plurality of Bragg gratings 261a, 261b, 261c disposed near a distal end 320 of a MMF in the core 350. Each of the Bragg gratings 261a, 261b, 261c has a different length (number of regions of different refractive index), with the result that each Bragg grating 261a, 261b, 261c has a different reflectance. This enables a checkerboard reflector to be implemented within the fibre that imposes a spatial encoding on the intensity of the reflected light from the modified regions (analogous to the approach of Gu).

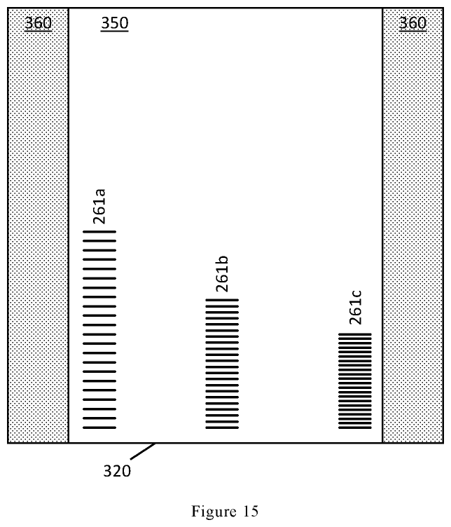

FIG. 15 shows a plurality of Bragg gratings 261a, 261b, 261c disposed near a distal end 320 of a MMF in the core 350. Each of the Bragg gratings 261a, 261b, 261c has a different spatial period, with the result that each Bragg grating 261a, 261b, 261c has a different Bragg wavelength (at which it will have maximum reflectance). Different wavelength Bragg gratings may be used to simplify interrogation of multiple modified regions at the same time (by wavelength multiplexing), and also facilitates an approach to determining the TM that is similar to that disclosed in Gordon.

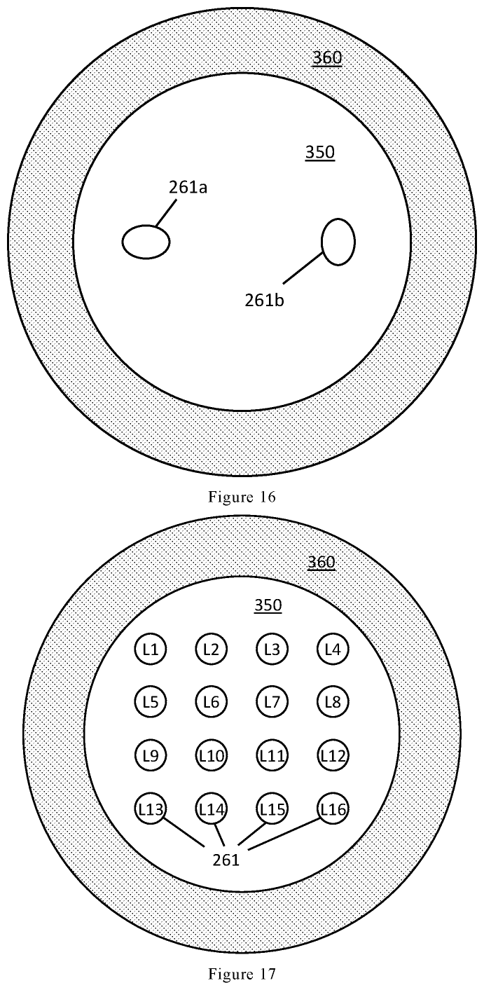

FIG. 16 shows a plurality of Bragg gratings 261a, 261b disposed near a distal end 320 of a MMF, in the core 350. Each of the Bragg gratings 261a, 261b has a different orientation of the regions of different refractive index, with the result that each Bragg grating 261a, 261b will have peak reflectance for a different polarisation of light. Polarisation specific Bragg gratings may be used to simplify interrogation of multiple modified regions at the same time (by polarisation multiplexing), and also facilitates an approach to determining the TM that is similar to that disclosed in Gordon.

FIG. 17 shows a plurality of modified regions L1-L16, disposed in a two dimensional array in the MMF fibre core 350 at a longitudinal location near the distal end of the MMF. The modified regions L1-L16 may comprise Bragg gratings, which may be configured to impose a spatial intensity modulation on the light reflected from the modified regions (e.g. by varying the length of the grating at each location).

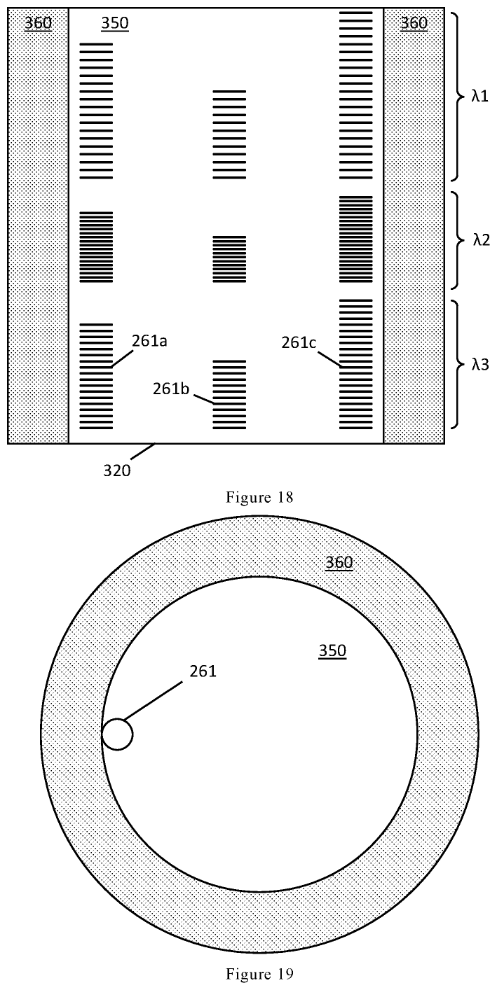

FIG. 18 shows a plurality of modified regions 261a, 261b, 261c at each of a plurality of longitudinal positions. The modified regions comprise Bragg gratings, and each longitudinal position comprises Bragg gratings with a particular grating period, so that each layer of Bragg gratings is configured to reflect a specific wavelength of light. Each longitudinal position may be provided with an array of Bragg gratings, as shown in FIG. 17. This embodiment may be applicable for determining the TM as disclosed in Gordon, with the stacked arrays of Bragg gratings analogous to the stacked metasurfaces in Gordon.

FIG. 19 shows an embodiment in which a single Bragg grating 261 is provided in the core 350 at the edge of the core 350, near the distal end of the fibre. In this embodiment the Bragg grating may serve as a guide star, to determine a TM that enables an isoplanatic patch, as described by Li.

Although embodiments with Bragg gratings have been described, some embodiments may employ fluorescent light from the modified regions, rather than reflected light. Since the fluoresced light will have a different wavelength, the sensing of the fluoresced light from the modified regions may be detected at the same time as sensing or imaging using the optical fibre (corrected by the TM determined from the fluoresced light from the modified regions). A secondary focus can be generated on the fluorescent modified region using holography, and temporal multiplexing between multiple fluorescent structures is also possible.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub combination.

The examples provided in the detailed description are intended to provide examples of the invention, not to limit its scope, which should be determined with reference to the accompanying claims.

1. A method of determining an optical transform imparted by a multimode optical fibre, wherein the multimode fibre comprises a proximal end, a distal end, and at least one modified region between the proximal end and distal end, the modified region configured to transmit light toward the proximal end in response to light propagating through the multimode optical fibre from the proximal end to the distal end, the method comprising:
coupling forward propagating light into the proximal end of the multimode optical fibre;
detecting, at the proximal end, backward propagating light transmitted from the at least one modified region in response to the forward propagating light; and
determining an optical transform from the detected backward propagating light.

2. The method of claim 1, wherein determining the optical transform comprises:
i) determining a transmission matrix of the multimode optical fibre, defining a relationship between an input field at a proximal facet and the resulting output field at a distal facet; and/or
ii) determining a correction matrix for correcting a predetermined transmission matrix.

3. The method of claim 1 or 2, wherein the at least one modified region comprises at least one fibre Bragg grating.

4. The method of any preceding claim 2, wherein the at least one modified region comprises at least one fluorescent colour centre

5. The method of any preceding claim, wherein the at least one modified region comprises one or more modified regions disposed in the fibre core and/or fibre cladding.

6. The method of any preceding claim, wherein there are a plurality of modified regions, with at least some of the modified regions at different lateral positions and/or at different longitudinal positions between the proximal and distal end.

7. The method of any preceding claim, wherein the at least one modified region comprises a plurality of fibre Bragg gratings, and at least some of the fibre Bragg gratings have different: period, reflectivity and/or orientation/polarisation

8. The method of any preceding claim, wherein the at least one modified region comprises a one or more chirped fibre Bragg gratings.

9. The method of any preceding claim, wherein the forward propagating light is a first forward propagating light field and the backward propagating light is a first backward propagating light field, and the method further comprises:
transmitting a second forward propagating light field for sensing/imaging a scene adjacent to the distal end;
detecting a second backward propagating light field transmitted through the fibre resulting from the second forward propagating light field, the method further comprising using the optical transform determined from the first backward propagating light field to correct an image formed using the second backward propagating light field.

10. The method of claim 9, wherein correcting the image comprises controlling an active optical element to modify the second forward propagating light field.

11. The method of claim 10, wherein the active optical element comprises a spatial light modulator configured to modify the spatial distribution of phase of the second forward propagating light field

12. The method of claim 10 or 11, wherein the method comprises performing point scanning microscopy using a plurality of second forward propagating light fields.

13. The method of claim 9, wherein correcting the image comprises computationally reconstructing an image from the results of detecting the second backward propagating light field.

14. The method of any of claims 9 to 13, wherein the first forward propagating light field and the second forward propagating light field and/or the first backward propagating and the second backward propagating light field are multiplexed, so that the optical transform can be updated without interrupting imaging of the scene.

15. The method of claim 14, wherein the multiplexing comprises wavelength multiplexing and/or temporal multiplexing.

16. The method of any preceding claim, wherein the modified region is formed by laser machining

17. The method of claim 16, wherein the laser micromachining is performed using adaptive optics, which modify wavefront properties of a laser system to counteract the effects of aberration on laser focus

18. The method of any preceding claim, wherein determining on optical transform comprises determining a correction matrix to take account of fibre deformation, wherein determining the optical transform comprises multiplying the correction matrix with an uncorrected transmission matrix.

19. The method of any preceding claim, wherein the uncorrected transmission matrix is determined by detecting, at the distal end of the fibre, forward propagating light coupled into the optical fibre at the proximal end of the fibre.

20. The method of any preceding claim, further comprising correcting the transmission matrix for temperature determined from a modified region that comprises a fibre Bragg grating.

21. Apparatus for obtaining information for correcting an optical transform imparted by a multimode optical fibre, comprising:
a multimode fibre comprising
a proximal end,
a distal end, and
at least one modified region between the proximal end and distal end, the modified region configured to transmit light toward the proximal end in response to light propagating through the multimode optical fibre from the proximal end to the distal end,
a light source coupled to the proximal end and configured to transmit forward propagating light into the optical fibre;
a detector coupled to the proximal end and configured to detect backward propagating light transmitted from the at least one modified region in response to the forward propagating light

22. The apparatus of claim 21, further comprising:
a processor configured to determine the optical transform from the detected backward propagating light.

23. The apparatus of claim 19 or 20, wherein the apparatus is configured to perform the method of any preceding claim.

24. An apparatus according to any of claims 21 to 23, wherein the forward propagating light is a first forward propagating light field and the backward propagating light is a first backward propagating light field; and
the light source is configured to transmit a second forward propagating light field for imaging a scene adjacent to the distal end;
the detector is configured to detect a second backward propagating light field transmitted through the fibre resulting from the second forward propagating light field; and
the apparatus is configured to use the detected backward propagating light field to correct an image formed using the second backward propagating light field.

25. The apparatus of claim 24, wherein the apparatus is configured to use an optical transform determined from the detected first backward propagating light field to correct the image formed using the second backward propagating light field.

26. An endoscope comprising the apparatus of claim 22 or 23.
